(19) United States
(12) Patent Application Publication
(10) Pub. No.: US 2026/0234675 A1
(43) Pub. Date: Aug. 13, 2026

(54) REDUCTION IN ACETATE PRODUCED BY YEAST WITH REDUCED EXPRESSION OF RSF2 OR TDA9

(71) Applicant: DANISCO US INC., Palo Alto, CA (US)

(72) Inventors: Courtney Bement, West Grove, PA (US); Zhongqiang Chen, Wilmington, DE (US); Xiaochun Fan, West Chester, PA (US); Daniel Joseph Macool, Wilmington, DE (US); Min Qi, Hockessin, DE (US); Yehong Jamie Wang, Wilmington, DE (US); Zhixiong Xue, Wilmington, DE (US); Quinn Qun Zhu, West Chester, PA (US)

(21) Appl. No.: 18/703,916

(22) PCT Filed: Oct. 26, 2022
(86) PCT No.: PCT/US2022/047796

§ 371 Date: Apr. 23, 2024

Related U.S. Application Data
(60) Provisional application No. 63/271,862, filed on Oct. 26, 2021.

(51) Int. Cl. C12P 7/06; C12N 15/52

(52) U.S. Cl. CPC C12P 7/06; C12N 15/52

(57) ABSTRACT

Described are compositions and methods relating to modified yeast with disrupted RSF2 or TDA9 genes. The yeast produces a decreased amount of acetate compared to otherwise identical parental cells. Such yeast is particularly useful for large-scale ethanol production from starch substrates where acetate is an undesirable end product.

### CROSS-REFERENCE TO RELATED APPLICATIONS

The present application is a U.S. National Stage Application under 35 U.S.C. § 371 of International Application No. PCT/US2022/047796, filed Oct. 26, 2022, which claims the benefit of U.S. Application No. 63/271,862, filed Oct. 26, 2021, each of which is hereby incorporated by reference in its entirety.

### TECHNICAL FIELD

The present compositions and methods relate to modified yeast with disrupted RSF2 or TDA9 genes. The yeast produces a decreased amount of acetate compared to parental cells. Such yeast is particularly useful for large-scale ethanol production from starch substrates, where acetate in an undesirable by-product.

### INCORPORATION BY REFERENCE OF THE SEQUENCE LISTING

The present application is being filed with a Sequence Listing in electronic format. The Sequence Listing is provided as a file entitled NB41891USPCT_SEQ_Listing.xml, created on Apr. 21, 2026, which is 9,811 bytes in size. The information in the electronic format of the Sequence Listing is incorporated by reference in its entirety.

### BACKGROUND

First-generation yeast-based ethanol production converts sugars into fuel ethanol. The annual fuel ethanol production by yeast is about 90 billion liters worldwide (Gombert, A. K. and van Maris. A. J. (2015) Curr. Opin. Biotechnol. 33:81-86). It is estimated that about 70% of the cost of ethanol production is the feedstock. Since the production volume is so large, even small yield improvements have massive economic impact across the industry.

Ethanol production in engineered yeast cells with a heterologous phosphoketolase (PKL) pathway is higher than in a parental strain without a PKL pathway (see, e.g., Miasnikov et al. WO2015148272). The PKL pathway consists of phosphoketolase (PKL) and phosphotransacetylase (PTA) to channel carbon flux away from the glycerol pathway and toward the synthesis of acetyl-coA. Two supporting enzymes, acetaldehyde dehydrogenase (AADH) and acetyl-coA synthase (ACS), can help the PKL pathway be more effective.

Unfortunately, the engineered strains also produce more acetate than the parental yeast. Acetate is not a desirable by-product as it has negative effects on yeast growth and fermentation. In addition, acetate reduces the pH of left-over water from fermentation and distillation, referred to as backset, which is typically reused for liquefaction of a subsequent batch of substrate. As a result, ethanol producers must adjust the pH of the backset (or liquefact) or increase the amount of fresh water used for liquefaction.

The need exists to control the amount of acetate produced by yeast, particularly engineered yeast that tend to produce an increased amount of acetate.

### SUMMARY

The present compositions and methods relate to modified yeast with reduced production of functional RSF2 or TDA9 polypeptides. Aspects and embodiments of the compositions and methods are described in the following, independently-numbered paragraphs.
1. In one aspect, modified yeast cells derived from parental yeast cells are provided, the modified cells comprising a genetic alteration that causes the modified cells to produce a decreased amount of RFS2 and/or TDA9 polypeptides compared to the parental cells, wherein the modified cells produce during fermentation a decreased amount of acetate compared to the amount of acetate produced by the parental cells under identical fermentation conditions.
2. In some embodiments of the modified cells of paragraph 1, the genetic alteration comprises the disruption of a nucleic acid capable of directing the expression of a RFS2 and/or TDA9 polypeptides compared to the level in the parental cells.
3. In some embodiments, the modified cells of paragraph 1 or 2 further comprise a genetic alteration that causes the modified cells to produce an increased amount of MIG3 polypeptides compared to the amount in parental cells.
4. In some embodiments of the modified cells of any of paragraphs 1-3, the cells further comprise one or more genes of the phosphoketolase pathway.
5. In some embodiments of the modified cells of paragraph 4, the genes of the phosphoketolase pathway are selected from the group consisting of phosphoketolase, phosphotransacetylase and acetylating acetyl dehydrogenase.
6. In some embodiments of the modified cells of any of paragraphs 1-5, the cells further comprise an exogenous gene encoding a carbohydrate processing enzyme.
7. In some embodiments, the modified cells of any of paragraphs 1-6 further comprise an alteration in the glycerol pathway and/or the acetyl-CoA pathway.
8. In some embodiments, the modified cells of any of paragraphs 1-7 further comprise an alternative pathway for making ethanol.
9. In some embodiments of the modified cells of any of paragraphs 1-8, the cells are of a Saccharomyces spp.
10. In another aspect, a method for decreasing the production of acetate from yeast cells grown on a carbohydrate substrate is provided, comprising: introducing into parental yeast cells a genetic alteration that that decreases the production of functional RSF2 and/or TAD9 polypeptides compared to the amount produced in the parental cells.
11. In some embodiments, the method of paragraph 10 further comprises introducing into the parental yeast cells a genetic alteration that increases the production of MIG3 polypeptides compared to the amount produced in the parental cells.
12. In some embodiments of the method of paragraph 10 or 11, the decrease in acetate production is at least 5%, at least 10%, at least 15%, at least 20%, at least 25%, at least 30%, at least 35%, at least 40%, or more.

These and other aspects and embodiments of present modified cells and methods will be apparent from the description, including any accompanying Drawings/Figures.

### DETAILED DESCRIPTION

### I. Definitions

Prior to describing the present yeast and methods in detail, the following terms are defined for clarity. Terms not defined should be accorded their ordinary meanings as used in the relevant art.

As used herein, the term “alcohol” refers to an organic compound in which a hydroxyl functional group (—OH) is bound to a saturated carbon atom.

As used herein, the terms “yeast cells,” “yeast strains,” or simply “yeast” refer to organisms from the phyla Ascomycota and Basidiomycota. Exemplary yeast is budding yeast from the order Saccharomycesles. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “engineered yeast cells,” “variant yeast cells,” “modified yeast cells,” or similar phrases, refer to yeast that include genetic modifications and characteristics described herein. Variant/modified yeast do not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins,” or “homologs.” Such proteins can be derived from organisms of different genera and/or species, or different classes of organisms (e.g., bacteria and fungi), or artificially designed. Related proteins also encompass homologs determined by primary sequence analysis, determined by secondary or tertiary structure analysis, or determined by immunological cross-reactivity, or determined by their functions.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity (ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, WI); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype. The term “allele” is generally preferred when an organism contains more than one similar genes, in which case each different similar gene is referred to as a distinct “allele.”

As used herein, “constitutive” expression refers to the production of a polypeptide encoded by a particular gene under essentially all typical growth conditions, as opposed to “conditional” expression, which requires the presence of a particular substrate, temperature, or the like to induce or activate expression.

As used herein, the term “expressing a polypeptide” and similar terms refers to the cellular process of producing a polypeptide using the translation machinery (e.g., ribosomes) of the cell.

As used herein, “over-expressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes a promoter, and amino acid coding region and a terminator (i.e., promoter::amino acid coding region::terminator) and other nucleic acid sequence needed to allow the encoded polypeptide to be produced in a cell. Expression cassettes can be exogenous (i.e., introduced into a cell) or endogenous (i.e., extant in a cell).

As used herein, the terms “fused” and “fusion” with respect to two DNA fragments, such as a promoter and the coding region of a polypeptide refer to a physical linkage causing the two DNA fragments to become a single molecule.

As used herein, the terms “wild-type” and “native” are used interchangeably and refer to genes, proteins or strains found in nature, or that are not intentionally modified for the advantage of the presently described yeast.

As used herein, the term “protein of interest” refers to a polypeptide that is desired to be expressed in modified yeast. Such a protein can be an enzyme, a substrate-binding protein, a surface-active protein, a structural protein, a selectable marker, or the like, and can be expressed. The protein of interest is encoded by an endogenous gene or a heterologous gene (i.e., gene of interest”) relative to the parental strain. The protein of interest can be expressed intracellularly or as a secreted protein.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using CRISPR, RNAi, antisense, or any other method that abolishes gene expression. A gene can be disrupted by deletion or genetic manipulation of non-adjacent control elements. As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements. Deletion of a gene also refers to the deletion a part of the coding sequence, or a part of promoter immediately or not immediately adjacent to the coding sequence, where there is no functional activity of the interested gene existed in the engineered cell.

As used herein, the terms “genetic manipulation,” “genetic alteration”, “genetic engineering”, and similar terms are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a binding activity, a surface-active property, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, altering signal transduction to turn on gene transcription or translation inside the cell, and combinations, thereof.

As used herein, “attenuation of a pathway” or “attenuation of the flux through a pathway,” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth and production process in the presence of oxygen.

As used herein, “anaerobic fermentation” refers to growth and production in the absence of oxygen.

As used herein, the expression “end of fermentation” refers to the stage of fermentation when the economic advantage of continuing fermentation to produce a small amount of additional alcohol is exceeded by the cost of continuing fermentation in terms of fixed and variable costs. In a more general sense, “end of fermentation” refers to the point where a fermentation will no longer produce a significant amount of additional alcohol, i.e., no more than about 1% additional alcohol.

As used herein, the expression “carbon flux” refers to the rate of turnover of carbon molecules through a metabolic pathway. Carbon flux is regulated by enzymes involved in metabolic pathways, such as the pathway for glucose metabolism and the pathway for maltose metabolism.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:
following meanings unless otherwise specified:
° C. degrees Centigrade
AA α-amylase
AADH acetaldehyde dehydrogenases
bp base pairs
DNA deoxyribonucleic acid
ds or DS dry solids
EC enzyme commission
EtOH ethanol
g or gm gram
g/L grams per liter
GA glucoamylase
H₂O water
HPLC high performance liquid chromatography
hr or h hour
kg kilogram
M molar
mg milligram
min minute
mL or ml milliliter
mM millimolar
N normal
nm nanometer
PCR polymerase chain reaction
PKL phosphoketolase
ppm parts per million
PTA phosphotransacetylase
Δ relating to a deletion
μ g microgram
μL and μl microliter
μM micromolar

### II. Modified Yeast Cells with Reduced Expression of Functional RSF2 or TDA9

RSF2 (YJR127C) is a zinc-finger protein; involved in transcriptional control of both nuclear and mitochondrial genes, many of which specify products required for glycerol-based growth, respiration, and other functions. RSF2 has a paralog, TDA9 (YML081W), that appears to have arisen from whole genome duplication. TDA9 relocalizes from nucleus to cytoplasm in response to DNA replication stress.

Applicants have discovered that yeast cells with reduced expression of functional RSF2 or TDA9 polypeptides produce less acetate than otherwise identical parental cells. Decreased acetate is desirable as acetate adversely affects yeast growth and fermentation and additionally results in backset that has a lower than desirable pH, requiring pH adjustment or the use of more fresh water to dilute the backset.

Reduction in the amount of functional RSF2 and/or TDA9 polypeptides can result from disruption of a gene encoding these polypeptide (i.e., YJR127C or YML081W, respectively) present in the parental strain. Because disruption of a YJR127C or YML081W gene is a primary genetic determinant for conferring the decreased acetate-production-phenotype to the modified cells, in some embodiments the modified cells need only comprise a disrupted YJR127C or YML081W gene, while all other genes can remain intact. In other embodiments, the modified cells can optionally include additional genetic alterations compared to the parental cells from which they are derived. While such additional genetic alterations are not necessary to confer the described phenotype, they may confer other advantages to the modified cells.

Disruption of a YJR127C or YML081W gene can be performed using any suitable methods that substantially reduce expression of a function RSF2 and/or TDA9 polypeptides. Exemplary methods of disruption as are known to one of skill in the art include but are not limited to: complete or partial deletion of a YJR127C or YML081W gene, including complete or partial deletion of, e.g., a RSF2 or TDA9-coding sequence, the promoter, the terminator, an enhancer, or another regulatory element; and complete or partial deletion of a portion of the chromosome that includes any portion of a YJR127C or YML081W gene.

Particular methods for disrupting a YJR127C or YML081W gene include making nucleotide substitutions or insertions in any portion of a YJR127C or YML081W gene, e.g., a RSF2 or TDA9-coding sequence, the promoter, the terminator, an enhancer, or another regulatory element. Preferably, deletions, insertions, and/or substitutions (collectively referred to as mutations) are made by genetic manipulation using sequence-specific molecular biology techniques, as opposed to by chemical mutagenesis, which is generally not targeted to specific nucleic acid sequences. Nonetheless, chemical mutagenesis can, in theory, be used to disrupt a YJR127C or YML081W gene.

In some embodiments, the decrease in the amount of functional RSF2 or TDA9 polypeptide produced by the modified cells is a decrease of at least 30%, at least 40%, at least 50%, at least 60%, at least 70%, at least 80%, at least 90%, at least 95%, at least 98%, at least 99%, or more, compared to the amount of functional RSF2 or TDA9 polypeptide produced by otherwise identical parental cells growing under the same conditions.

In some embodiments, the decrease in acetate produced by the modified cells is a decrease of at least 1%, at least 2%, at least 3%, at least 4%, at least 5%, or more, compared to the amount of acetate produced by parental cells growing under the same conditions.

### III. Modified Yeast Cells with Reduced Expression of RSF2 or TDA9 and Over-Expressing MIG3

MIG3 is a Cys₂His₂ zinc finger protein that has a sequence similarity with two other transcription factors, MIG1 and MIG2. All three transcription factors share extensive amino acid identity (>70%) in their DNA binding domains. A recently study showed that MIG3 has a role in catabolite repression and an additional regulatory role upon ethanol exposure in some strains (Lewis, J. A. and Gasch, A. P. (2012) G3 2:1607-12).

Applicants recently discovered that yeast cells over-expressing MIG3 polypeptides produce decreased amounts of acetate compared to otherwise-identical parental cells (WO2021108464). Decreased acetate is desirable as acetate adversely affects yeast growth and fermentation and additionally results in backset that has a lower than desirable pH, requiring pH adjustment or the use of more fresh water to dilute the backset.

Here, Applicants, demonstrate that over-expression MIG3 polypeptides and decreased expression of RSF2 or TAD9 have an additive effect for acetate reduction in yeast, including those having an exogenous PKL pathway.

In some embodiments, the increase in the amount of MIG3 polypeptides produced by the modified cells is an increase of at least 20%, at least 30%, at least 40%, at least 50%, at least 70%, at least 100%, at least 150%, at least 200%, at least 500%, at least 1,000%, at least 2,000%, or more, compared to the amount of MIG3 polypeptides produced by parental cells grown under the same conditions.

In some embodiments, the decrease in acetate production by the modified cells is a decrease of at least 5%, at least 10%, at least 15%, at least 20%, at least 25%, at least 30%, at least 35%, at least 40%, or more, compared to the amount of acetate produced by parental cells grown under the same conditions.

Preferably, increased MIG3 expression is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

Particular methods of over-expression include but are not limited to (i) introducing an exogenous expression cassette for producing the polypeptide into a host cell, optionally in addition to an endogenous expression cassette, (ii) substituting an exogenous expression cassette with an endogenous cassette that allows the production of an increased amount of the polypeptide, (iii) modifying the promoter of an endogenous expression cassette to increase expression, and/or (iv) modifying any aspect of the host cell to increase the half-life of the polypeptide in the host cell.

### IV. Modified Yeast Cells with Genes of an Exogenous PKL Pathway

Reduced expression of RSF2 or TDA9, optionally with over-expression of MIG3, can be combined with expression of genes in the PKL pathway to reduce the production of elevated amounts of acetate that is associated with introducing an exogenous PKL pathway into yeast.

Engineered yeast cells having a heterologous PKL pathway have been previously described (WO2015148272). These cells express heterologous phosphoketolase (PKL), phosphotransacetylase (PTA) and acetylating acetyl dehydrogenase (AADH), optionally with other enzymes, to channel carbon flux away from the glycerol pathway and toward the synthesis of acetyl-CoA, which is then converted to ethanol. Such modified cells are capable of increased ethanol production in a fermentation process when compared to otherwise-identical parent yeast cells.

### V. Modified Yeast Cells with Other Mutations that Affect Alcohol Production

In some embodiments, in addition to expressing decreased amounts of RSF2 or TDA9, optionally with over-expression of MIG, and further optionally with a heterologous PKL pathway, the present modified yeast cells include additional beneficial modifications.

The modified cells may further include mutations that result in attenuation of the native glycerol biosynthesis pathway and/or reuse glycerol pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.). Methods to enhance the reuse glycerol pathway by over expression of glycerol dehydrogenase (GCY1) and dihydroxyacetone kinase (DAK1) to convert glycerol to dihydroxyacetone phosphate (Zhang et al. (2013) J. Ind. Microbiol. Biotechnol. 40:1153-60).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-COA. This partially reduces the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lacks a heterologous gene(s) encoding an acetylating acetaldehyde dehydrogenase, a pyruvate-formate lyase or both.

In some embodiments, the present modified yeast cells may further over-express a sugar transporter-like (STL1) polypeptide to increase the uptake of glycerol (see, e.g., Ferreira et al. (2005) Mol. Biol. Cell. 16:2068-76; Dušková et al. (2015) Mol. Microbiol. 97:541-59 and WO 2015023989 A1) to increase ethanol production and reduce acetate.

In some embodiments, the present modified yeast cells further include a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, over-expression, and/or substitution in one or more endogenous polynucleotides encoding FRA2, ALD6, ADH1, GPD2, BDH1, DLS1, DPB3, CPR1, MAL23C, MNN4, PAB1, TMN2, HAC1, PTC1, PTC2, OSM1, GIS1, CRZ1, HUG1, GDS1, CYB2P, SFC1, MVB12, LDB10, C5SD, GIC1, GIC2, YMR226C, PHO13, ADH5 MIG1, MIG2, MIG3, JID1, KGD2, ARG7, LEU4, MET2, DAL7 and ISN1.

### VI. Modified Yeast Cells with Other Beneficial Mutations

In some embodiments, in addition to expressing decreased amounts of RSF2 or TDA9, optionally with over-expression of MIG, further optionally with a heterologous PKL pathway, and still further optionally in combination with other genetic modifications that benefit alcohol production, the present modified yeast cells further include any number of additional genes of interest encoding proteins of interest. Additional genes of interest may be introduced before, during, or after genetic manipulations that result in the increased production of active MIG3 polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VII. Use of the Modified Yeast for Increased Alcohol Production

The present compositions and methods include methods for increasing alcohol production and/or reducing glycerol production, in fermentation reactions. Such methods are not limited to a particular fermentation process. The present engineered yeast is expected to be a “drop-in” replacement for convention yeast in any alcohol fermentation facility. While primarily intended for fuel alcohol production, the present yeast can also be used for the production of potable alcohol, including wine and beer.

### VIII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### IX. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present yeast strains and methods will be apparent to the skilled person in view of the present description. The following examples are intended to further illustrate, but not limit, the compositions and methods.

### EXAMPLES

### Example 1

### Materials and Methods

### Liquefact Preparation:

Liquefact (corn mash slurry) was prepared by adding 600 ppm of urea, 0.124 SAPU/g ds acid fungal protease, 0.33 GAU/g ds variant Trichoderma reesei glucoamylase and 1.46 SSCU/g ds Aspergillus kawachii α-amylase, adjusted to a pH of 4.8 with sulfuric acid.

### Serum Vial Assays:

2 mL of YPD in 24-well plates were inoculated with yeast cells and the cultures allowed to grow overnight to an OD between 25-30. 2.5 mL liquefact was transferred to serum vials (Chemglass, Catalog No. CG-4904-01) and yeast was added to each vial to a final OD of about 0.4-0.6. The lids of the vials were installed and punctured with needle (BD, Catalog No. 305111) for ventilation (to release CO₂), then incubated at 32° C. with shaking at 200 RPM for 65 hours.

### AnKom Assays:

300 μL of concentrated yeast overnight culture was added to each of a number ANKOM bottles filled with 50 g prepared liquefact (see above) to a final OD of 0.3. The bottles were then incubated at 32° C. with shaking at 150 RPM for 65 hours.

### HPLC Analysis:

Samples of the cultures from serum vials and AnKom assays were collected in Eppendorf tubes by centrifugation for 12 minutes at 14,000 RPM. The supernatants were filtered using 0.2 μM PTFE filters and then used for HPLC (Agilent Technologies 1200 series) analysis with the following conditions: Bio-Rad Aminex HPX-87H columns, running temperature of 55 C. 0.6 ml/min isocratic flow 0.01 N H₂SO₄, 2.5 μl injection volume. Calibration standards were used for quantification of the of acetate, ethanol, glycerol, glucose and other molecules. All values are reported in g/L.

### RNA-Seq Analysis:

RNA was prepared from individual samples according to the TRIzol method (Life-Tech, Rockville, MD). The RNA was then cleaned up with Qiagen RNeasy Mini Kit (Qiagen, Germantown, MD). The cDNA from total mRNA in individual samples was generated using Applied Biosystems High Capacity cDNA Reverse Transcription kit (Thermo Fisher Scientific, Wilmington, Delaware). The prepared cDNA of each sample was sequenced using the shotgun method, and then quantified with respect to individual genes. The results are reported as reads per kilobase ten million transcripts (RPK10M), and used to quantify the amount of each transcript in a sample.

### Example 2

### Generation of Strains for Overexpressing MIG3 and Disrupting TDA9

The MIG3 gene (YER028C) of Saccharomyces cerevisiae was codon optimized and synthesized as MIG3s. An over expression including SUI3Pro::MIG3s::GPDIter was introduced at TAD9 locus, resulting in the disruption (in this case knock out) of the TDA9 gene in strains FERMAX™ Gold (Martrex, Inc., Chaska, MN, USA; herein FG) and FG-PKL (WO2015148272) using CRISPR Cas-9 technology. This single step method was used to express MIG3 and delete TAD9 gene simultaneously.

The amino acid sequence of the MIG3 polypeptide is shown,
below, as SEQ ID NO: 1:
MNYLRDRFPPDNDQRPFRCEICSRGFHRLEHKKRHVRTHTGEKPHKCTVQGCPKSFSRSDEL
KRHLRTHTKGVQRRRIKSKGSRKTVVNTATAAPTTFNENTGVSLTGIGQSKVPPILISVAQN
CDDVNIRNTGNNNGIVETQAPAILVPVINIPNDPHPIPSSLSTTSITSIASVYPSTSPFQYL
KSGFPEDPASTPYVHSSGSSLALGELSSNSSIFSKSRRNLAAMSGPDSLSSSKNQSSASLLS
QTSHPSKSFSRPPTDLSPLRRIMPSVNTGDMEISRTVSVSSSSSSLTSVTYDDTAAKDMGMG
IFFDRPPVTQKACRSNHKYKVNAVSRGRQHERAQFHISGDDEDSNVHRQESRASNTSPNVSL
PPIKSILRQIDNFNSAPSYFSK
The nucleic acid sequence of the MIG3-coding region is shown,
below, as SEQ ID NO: 2:
ATGAATTACCTGCGAGATAGATTTCCTCCGGATAATGACCAAAGACCCTTTAGATGTGAAAT
TTGTTCACGAGGTTTCCACAGACTTGAACATAAAAAAAGGCACGTAAGAACGCACACTGGCG
AGAAGCCTCACAAATGTACCGTTCAGGGCTGTCCGAAAAGCTTCAGCCGAAGCGATGAACTA
AAAAGACATTTGAGGACACATACTAAAGGCGTCCAAAGGCGCAGAATAAAATCCAAGGGCTC
GCGAAAAACCGTTGTGAATACTGCTACCGCCGCCCCTACCACCTTCAATGAAAACACTGGTG
TTTCGCTCACGGGGATAGGTCAATCTAAAGTGCCACCTATTCTTATCTCCGTTGCTCAGAAT
TGCGATGACGTGAATATACGAAATACTGGAAATAATAATGGCATTGTGGAGACACAGGCACC
TGCAATTTTAGTGCCTGTGATAAATATTCCAAATGACCCTCATCCGATTCCAAGTAGCCTCT
CCACTACTTCTATCACCTCCATTGCATCAGTATATCCCTCTACTTCTCCATTCCAGTACCTG
AAAAGCGGGTTTCCTGAAGATCCTGCATCTACACCGTATGTACATTCGTCCGGAAGTTCTTT
AGCCCTGGGTGAATTGTCTTCAAACTCCTCTATATTTTCGAAATCTAGGAGGAATTTGGCCG
CCATGAGTGGTCCTGATTCTTTGAGTAGTTCTAAAAACCAATCCAGTGCTTCGCTTCTTTCT
CAAACTTCACATCCATCAAAGAGCTTTTCAAGACCGCCAACAGACTTAAGTCCTCTGCGAAG
AATCATGCCTTCTGTAAACACAGGAGACATGGAAATTTCAAGGACAGTATCCGTTTCGAGCA
GTTCATCATCACTCACTTCTGTTACGTATGATGACACCGCGGCTAAAGACATGGGCATGGGA
ATATTTTTTGATAGGCCACCTGTAACACAGAAAGCTTGCAGGAGCAATCATAAGTACAAGGT
TAATGCTGTTAGCAGAGGGAGACAACATGAAAGGGCACAATTTCATATATCTGGAGATGATG
AGGACAGTAACGTTCACCGCCAAGAATCAAGAGCATCCAACACAAGTCCCAATGTATCATTG
CCTCCGATAAAGAGCATTTTGCGACAAATTGATAATTTCAACAGTGCTCCTTCTTACTTCAG
TAAATAA
The nucleic acid sequence of the SUI3 (YPL237W) promoter
(abbreviated above as GPD1ter, above) is shown, below, as
SEQ ID NO: 3:
CTTTTGTTAATGAGGTGAACAAAACAGGCAACACGGCTTTACATTGGGCGTCGTTGAATGG
CAAATTAGACGTGGTCAAGCTACTGTGTGATGAATATGAGGCAGACCCCTTTATTAGAAAC
AAATTCGGCCACGATGCTATCTTTGAGGCCGAGAACAGCGGGAAGGAAGAAGTGGAAACAT
ACTTTTTGAAGAAGTATGATGTCGAACCTGAAGATGATGAAGAAGACACACAAACTGAGGG
CAAGAATTCGGTCCAAATCACAAAGGGTACAGAAATTGAACAAGTCACCAAGGAAGCCACC
GAGGCTTTAAGAGAAGAAACCGAGAAACTGAATATAAATAAAGACTAAAGTAAAGAGCTTG
TTTTCTTCGTGGAATAAAAGCCGTAATATCCATTGAGCATGATATTTTATTTCTTGGTACC
GGAGAAGATAAAAGGATGGAACCAGCAGAAATGCATTAATAAATACAAAAAATTTAGAAAA
GAGTTACAGTAATAATGTATATTTCTCTCACAAACTAAGTAACCGCTTCAAAAGCGTATAT
TTTGAATGCATTGAACTTTCCATTTCATTACCCGCAGAGCCGGAGTCCTCATCAACGACGG
AGTGAAAAATTTTGTAATTGCGAGAAAAAGTGAAATTGATGGAAAAAAAGAAAAAGAAAGT
AGAAGAAGACATTATAAGAGAGACTAGGAAACTTCTTGCACATCAACCGAAAAGCGCCTAG
GCAACCAGTCATATAATAAGCACGCACGAG
The nucleic acid sequence of the GPD1 (YDL022W) terminator
(abbreviated above as SUI3Pro) shown, below, as SEQ ID NO: 4:
ATTTATTGGAGAAAGATAACATATCATACTTTCCCCCACTTTTTTCGAGGCTCTTCTATAT
CATATTCATAAATTAGCATTATGTCATTTCTCATAACTACTTTATCACGTTAGAAATTACT
TATTATTATTAAATTAATACAAAATTTAGTAACCAAATAAATATAAATAAATATGTATATT
TGAATTTTAAAAAAAAAATCCTATAGAGCAAAAGGATTTTCCATTATAATATTAGCTGTAC
ACCTCTTCCGCATTTTTTGAGGGTGGTTACAACACCACTCATTCAGAGGCTGTCGGCACAG
TTGCTTCTAGCATCTGGCGTCCGTATGTATGGGTGTATTTTAAATAA
The nucleic acid sequence of gRNA cassette is shown, below,
as SEQ ID NO: 5 (the underlined sequence is the gRNA-sequence
specific for TDA9):
TCATGCGGCCGCCCCTCACTAAAGGGAACAAAAGCTGGAGCTTCTTTGAAAAGATAATGTAT
GATTATGCTTTCACTCATATTTATACAGAAACTTGATGTTTTCTTTCGAGTATATACAAGGT
GATTACATGTACGTTTGAAGTACAACTCTAGATTTTGTAGTGCCCTCTTGGGCTAGCGGTAA
AGGTGCGCATTTTTTCACACCCTACAATGTTCTGTTCAAAAGATTTTGGTCAAACGCTGTAG
AAGTGAAAGTTGGTGCGCATGTTTCGGCGTTCGAAACTTCTCCGCAGTGAAAGATAAATGAT
CGGCAGCACTTGAGCTCGCGGGGTTTTAGAGCTAGAAATAGCAAGTTAAAATAAGGCTAGTC
CGTTATCAACTTGAAAAAGTGGCACCGAGTCGGTGGTGCTTTTTTTGTTTTTTATGTCTGAG
CTCACGT

### Example 3

### Generation of Strains for Overexpressing MIG3 and Disrupting RSF2

Similar to the procedure described in Example 2, a MIG3 expression cassette consisting of SUI3Pro::MIG3s::GPDIter was introduced at the RSF2 locus, resulting in the expression of MIG3 and simultaneous disruption of RSF2 in strains FG and FG-PKL, using CRISPR Cas-9 technology. The amino acid sequences of the MIG3 polypeptide, MIG3-coding region, SUI3 promoter, and GPD1 terminator are shown, above, in Example 2. The nucleic acid sequence of gRNA cassette is shown, below, as SEQ ID NO: 6 (the underlined sequence is the gRNA-sequence specific for RSF2):

TCATGCGGCCGCCCCTCACTAAAGGGAACAAAAGCTGGAGCTTCTTTG
AAAAGATAATGTATGATTATGCTTTCACTCATATTTATACAGAAACTT
GATGTTTTCTTTCGAGTATATACAAGGTGATTACATGTACGTTTGAAG
TACAACTCTAGATTTTGTAGTGCCCTCTTGGGCTAGCGGTAAAGGTGC
GCATTTTTTCACACCCTACAATGTTCTGTTCAAAAGATTTTGGTCAAA
CGCTGTAGAAGTGAAAGTTGGTGCGCATGTTTCGGCGTTCGAAACTTC
TCCGCAGTGAAAGATAAATGATCGCTCAATAGGCGGCTGGCATAGTTT
TAGAGCTAGAAATAGCAAGTTAAAATAAGGCTAGTCCGTTATCAACTT
GAAAAAGTGGCACCGAGTCGGTGGTGCTTTTTTTGTTTTTTATGTCTG
AGCTCACGT

### Example 4

### Performance of Strains Over-Expressing MIG3 with Disrupted RSF2 or TDA9

Table 1 summarizes Ankom results of the performance of FG strains over-expressing expressing MIG3 (FG-MIG3; WO2021108464), being disrupted for RSF2 or TDA9 (FG-RSF2A and FG-TAD9A, respectively) or both (FG-MIG3/RSF2A and FG-MIG3/TDA9A, respectively), were tested for glucose, glycerol, acetic acid and ethanol production, as described in Example 1. Acetate was reduced by 18-20% with the FG strain with TAD9A or RSF2A without overexpression of MIG3. Acetate was reduced by about 32% with the FG strain only overexpressing MIG3. Acetate was reduced by about 42% in the strain with the MIG3/RSF2A genotype.

TABLE 1
Performance of FG strains over-expressing MIG3 with or without the TDA9 or RSF2 disruption

| | | | | | Ethanol | Acetate |
|---|---|---|---|---|---|---|
| Strain | Glucose | Glycerol | Ethanol | Acetate | (% of FG) | (% of FG) |
| FG | 1.27 | 15.18 | 142.64 | 1.31 | -1- | -1- |
| FG-TAD9Δ | 0.94 | 14.94 | 144.09 | 1.07 | 1.010 | 0.82 |
| FG-RSF2Δ | 0.52 | 15.18 | 143.05 | 1.05 | 1.003 | 0.80 |
| FG-MIG3 | 0.82 | 14.83 | 143.24 | 0.90 | 1.004 | 0.68 |
| FG-MIG3/TAD9Δ | 1.63 | 14.44 | 143.50 | 0.89 | 1.006 | 0.68 |
| FG-MIG3/RSF2Δ | 0.56 | 15.19 | 142.95 | 0.76 | 1.002 | 0.58 |

### Example 5

### Performance of PKL Strain Over-Expressing MIG3 with Disrupted RSF2 or TDA9

Table 2 summarizes Ankom results of the performance of FG-PKL strains over-expressing MIG3 with or without the RSF2 or TDA9 disruptions. Strain nomenclature is similar to that used in Example 4. FG-PKL-MIG3 strains were constructed in a manner similar to that described in WO2021108464. Acetate was reduced by about 33% with MIG3/TAD9A and about 38% with MIG3/RSF2A compared to the parental PKL strain. Acetate was reduced only by 15-20% in strains with only the RSF2 or TAD9 disruption, or only MIG3 overexpression. Ethanol titer was increased slightly in FG-PKL strains with MIG3 overexpression in combination with the RSF2 or TDA9 disruption.

TABLE 2
Performance of FG-PKL strains over-expressing MIG3 with or without the TDA9 or RSF2 disruption

| | | | | | Ethanol (% | Acetate (% |
|---|---|---|---|---|---|---|
| Strain | Glucose | Glycerol | Ethanol | Acetate | FG-PKL) | FG-PKL) |
| FG-PKL | 4.04 | 13.14 | 142.97 | 1.94 | 1.000 | 1.00 |
| FG-PKL-TAD9Δ | 2.34 | 13.62 | 143.88 | 1.61 | 1.006 | 0.83 |
| FG-PKL- | 1.97 | 13.63 | 143.07 | 1.65 | 1.001 | 0.85 |
| RSF2LΔ | | | | | | |
| FG-PKL-MIG3 | 4.23 | 13.01 | 143.16 | 1.55 | 1.001 | 0.80 |
| FG-PKL- | 3.70 | 13.53 | 143.35 | 1.30 | 1.003 | 0.67 |
| MIG3/TAD9Δ | | | | | | |
| FG-PKL- | 1.81 | 13.84 | 143.70 | 1.20 | 1.005 | 0.62 |
| MIG3/RSF2Δ | | | | | | |

1. Modified yeast cells derived from parental yeast cells, the modified yeast cells comprising a genetic alteration that causes the modified yeast cells to produce a decreased amount of one or more of RFS2 and/or TDA9 polypeptides compared to the parental yeast cells, wherein the modified yeast cells produce during fermentation a decreased amount of acetate compared to the amount of acetate produced by the parental yeast cells under identical fermentation conditions.

2. The modified yeast cells of claim 1, wherein the genetic alteration comprises disruption of one or more of a nucleic acid capable of directing the expression of a RFS2 or a nucleic acid capable of directing the expression of a TDA9 polypeptides.

3. The modified yeast cells of claim 1, further comprising a genetic alteration that causes the modified yeast cells to produce an increased amount of MIG3 polypeptides compared to the parental yeast cells.

4. The modified yeast cells of claim 1, the further comprising one or more genes of the phosphoketolase pathway.

5. The modified yeast cells of claim 4, wherein the one or more genes of the phosphoketolase pathway are selected from the group consisting of phosphoketolase, phosphotransacetylase and acetylating acetyl dehydrogenase.

6. The modified yeast cells of claim 1, further comprising an exogenous gene encoding a carbohydrate processing enzyme.

7. The modified yeast cells of claim 1, further comprising an alteration in the glycerol pathway and/or the acetyl-CoA pathway.

8. The modified yeast cells of claim 1, further comprising an alternative pathway for making ethanol.

9. The modified yeast cells of claim 1, wherein the modified yeast cells are of a Saccharomyces spp.

10. A method for decreasing the production of acetate from yeast cells grown on a carbohydrate substrate, comprising: introducing into parental yeast cells a genetic alteration that that decreases the production of functional RSF2 and/or TAD9 polypeptides compared to the amount produced in the parental cells.

11. The method of claim 10, further comprising introducing into the parental yeast cells a genetic alteration that increases the production of MIG3 polypeptides compared to the amount produced in the parental cells.

12. The method of claim 10, wherein the decrease in acetate production is at least 5%, at least 10%, at least 15%, at least 20%, at least 25%, at least 30%, at least 35%, at least 40%, or more.

13. A method for producing alcohol comprising, fermenting a carbohydrate substrate with the modified yeast cells of claim 1.

14. The method of claim 13, wherein the modified yeast cells produce during fermentation a decreased amount of acetate compared to the amount of acetate produced by the parental yeast cells under identical fermentation conditions.

15. The method of claim 13, wherein the alcohol is ethanol.

16. The method of claim 13, wherein the carbohydrate substrate is corn starch, sugar cane, cassava, or molasses.


SEQUENCE LISTING (6 sequences)

SEQ ID NO: 1 - protein, 394 aa, Saccharomyces cerevisiae
MNYLRDRFPPDNDQRPFRCEICSRGFHRLEHKKRHVRTHTGEKPHKCTVQGCPKSFSRSDELKRHLRTHTKGVQRRRIKSKGSRKTVVNTATAAPTTFNENTGVSLTGIGQSKVPPILISVAQNCDDVNIRNTGNNNGIVETQAPAILVPVINIPNDPHPIPSSLSTTSITSIASVYPSTSPFQYLKSGFPEDPASTPYVHSSGSSLALGELSSNSSIFSKSRRNLAAMSGPDSLSSSKNQSSASLLSQTSHPSKSFSRPPTDLSPLRRIMPSVNTGDMEISRTVSVSSSSSSLTSVTYDDTAAKDMGMGIFFDRPPVTQKACRSNHKYKVNAVSRGRQHERAQFHISGDDEDSNVHRQESRASNTSPNVSLPPIKSILRQIDNFNSAPSYFSK

SEQ ID NO: 2 - DNA, 1185 bp, Synthetic construct
  source 1..1185: mol_type = other DNA
atgaattacctgcgagatagatttcctccggataatgaccaaagaccctttagatgtgaaatttgttcacgaggtttccacagacttgaacataaaaaaaggcacgtaagaacgcacactggcgagaagcctcacaaatgtaccgttcagggctgtccgaaaagcttcagccgaagcgatgaactaaaaagacatttgaggacacatactaaaggcgtccaaaggcgcagaataaaatccaagggctcgcgaaaaaccgttgtgaatactgctaccgccgcccctaccaccttcaatgaaaacactggtgtttcgctcacggggataggtcaatctaaagtgccacctattcttatctccgttgctcagaattgcgatgacgtgaatatacgaaatactggaaataataatggcattgtggagacacaggcacctgcaattttagtgcctgtgataaatattccaaatgaccctcatccgattccaagtagcctctccactacttctatcacctccattgcatcagtatatccctctacttctccattccagtacctgaaaagcgggtttcctgaagatcctgcatctacaccgtatgtacattcgtccggaagttctttagccctgggtgaattgtcttcaaactcctctatattttcgaaatctaggaggaatttggccgccatgagtggtcctgattctttgagtagttctaaaaaccaatccagtgcttcgcttctttctcaaacttcacatccatcaaagagcttttcaagaccgccaacagacttaagtcctctgcgaagaatcatgccttctgtaaacacaggagacatggaaatttcaaggacagtatccgtttcgagcagttcatcatcactcacttctgttacgtatgatgacaccgcggctaaagacatgggcatgggaatattttttgataggccacctgtaacacagaaagcttgcaggagcaatcataagtacaaggttaatgctgttagcagagggagacaacatgaaagggcacaatttcatatatctggagatgatgaggacagtaacgttcaccgccaagaatcaagagcatccaacacaagtcccaatgtatcattgcctccgataaagagcattttgcgacaaattgataatttcaacagtgctccttcttacttcagtaaataa

SEQ ID NO: 3 - DNA, 762 bp, Synthetic construct
  source 1..762: mol_type = other DNA
cttttgttaatgaggtgaacaaaacaggcaacacggctttacattgggcgtcgttgaatggcaaattagacgtggtcaagctactgtgtgatgaatatgaggcagacccctttattagaaacaaattcggccacgatgctatctttgaggccgagaacagcgggaaggaagaagtggaaacatactttttgaagaagtatgatgtcgaacctgaagatgatgaagaagacacacaaactgagggcaagaattcggtccaaatcacaaagggtacagaaattgaacaagtcaccaaggaagccaccgaggctttaagagaagaaaccgagaaactgaatataaataaagactaaagtaaagagcttgttttcttcgtggaataaaagccgtaatatccattgagcatgatattttatttcttggtaccggagaagataaaaggatggaaccagcagaaatgcattaataaatacaaaaaatttagaaaagagttacagtaataatgtatatttctctcacaaactaagtaaccgcttcaaaagcgtatattttgaatgcattgaactttccatttcattacccgcagagccggagtcctcatcaacgacggagtgaaaaattttgtaattgcgagaaaaagtgaaattgatggaaaaaaagaaaaagaaagtagaagaagacattataagagagactaggaaacttcttgcacatcaaccgaaaagcgcctaggcaaccagtcatataataagcacgcacgag

SEQ ID NO: 4 - DNA, 352 bp, Synthetic construct
  source 1..352: mol_type = other DNA
atttattggagaaagataacatatcatactttcccccacttttttcgaggctcttctatatcatattcataaattagcattatgtcatttctcataactactttatcacgttagaaattacttattattattaaattaatacaaaatttagtaaccaaataaatataaataaatatgtatatttgaattttaaaaaaaaaatcctatagagcaaaaggattttccattataatattagctgtacacctcttccgcattttttgagggtggttacaacaccactcattcagaggctgtcggcacagttgcttctagcatctggcgtccgtatgtatgggtgtattttaaataa

SEQ ID NO: 5 - DNA, 441 bp, Synthetic construct
  source 1..441: mol_type = other DNA
tcatgcggccgcccctcactaaagggaacaaaagctggagcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcggcagcacttgagctcgcggggttttagagctagaaatagcaagttaaaataaggctagtccgttatcaacttgaaaaagtggcaccgagtcggtggtgctttttttgttttttatgtctgagctcacgt

SEQ ID NO: 6 - DNA, 441 bp, Synthetic construct
  source 1..441: mol_type = other DNA
tcatgcggccgcccctcactaaagggaacaaaagctggagcttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgctcaataggcggctggcatagttttagagctagaaatagcaagttaaaataaggctagtccgttatcaacttgaaaaagtggcaccgagtcggtggtgctttttttgttttttatgtctgagctcacgt